(19) United States
(12) Patent Application Publication
(10) Pub. No.: US 2011/0184325 A1
(43) Pub. Date: Jul. 28, 2011

(54) ORTHOPEDIC APPARATUSES FOR MOBILIZING, STRETCHING AND PROTECTING THE SPINAL COLUMN

(76) Inventors: Kamran Behzadian, Sunnyvale, CA (US); Lorne Eltherington, Menlo Park, CA (US); Farida Behzadian, Tehran (IR)

(21) Appl. No.: 13/011,913

(22) Filed: Jan. 23, 2011

Related U.S. Application Data
(60) Provisional application No. 61/298,091, filed on Jan. 25, 2010.

(51) Int. Cl. A61F 5/00

(52) U.S. Cl. 602/19

(57) ABSTRACT

The described invention relates to devices for relief of spine pain and correcting posture. The device is a wearable frame equipped with a series of leaf or coil springs where the spring forces are adjusted so that the forces are applied parallel to the spine and are equal to or greater than the individual's body weight above the device. The compressed springs apply enough opposing force to transfer the downward force resulting from the weight to the body above the device to anatomy below the device while allowing normal spine motion relative to the body.

### CROSS REFERENCE

This application claims the benefit of U.S. Provisional Application No. 61/298,091, filed Jan. 25, 2010, entitled “Orthopedic Apparatus for Mobilizing and Stretching the Spinal Column” which application is incorporated herein by reference.

### FIELD OF THE INVENTION

The invention relates generally to orthopedic spinal bracing devices with adjustable forces in the form of an external wearable frame worn around the spine, that transfers the force applied by the weight of the human body above the device to the anatomy below; therefore, reducing or removing the axial pressure on the spine while allowing normal motions of the spine for everyday activities. The devices of this invention may be used on the cervical spine, the thoracolumbar spine, and may be used for protection of the neck in impact sports.

### BACKGROUND OF THE INVENTION

Spine pain may include pain generated in the cervical, thoracic, lumbar or a combination of multiple sections of the spine. As an example, lower back pain is a problem for a large percentage of people. Most back pain is generated in the lumbar portion of the spine due to the axial force generated by the weight of the upper body that is transferred through the spine structure. A compromise or deformity in the structure of the spine may apply pressure to a nerve in or around the spine and therefore cause pain.

Neck pain is also a problem for a large percentage of people. By age 55, about 95% of the population will experience some degenerative condition of the cervical spine that may cause neck pain and/or upper extremity pain commonly accompanied with weakness. These non-tumorous and non-infectious conditions may include; degenerative intervertebral discs, disc herniations, internal disc disruption, vertebral osteophytes or spur formation and spondylolisthesis, and they may potentially result in loss of disc space, height, encroachment on spinal nerve roots where they exit the spinal cord, or radiculopathy, regional spinal cord compression, myelopathy, or vertebral joint instability. Moreover, yearly work-injury-related neck and/or back pain may frequently affect as much as 15 to 20% of the workforce; for example, the 1990 annual cost of neck and lower back care in the United States reached $85 billion.

Most people who have severe spinal pain tend to lie on their backs in order to temporarily relieve their pain. By lying on their back, a patient removes the axial pressure on the spine because the body weight is no longer transferred through the spine structure. Most pain generated in the spine is due to the axial forces produced by gravity and the weight of the body that is transferred through the spine structure. A compromise or deformity in the structure of the spine may apply pressure to nerves in or around the organ and therefore cause pain.

Spinal traction is the technique of removing the weight of a portion of the body from the spinal axis. Traction is often followed by stretching the spinal column in order to relieve stress within the spine. This method can temporarily remove a large amount of pain experienced by people with spinal disorders; however, there is a downside to current traction methods, for the majority of the currently available traction devices are very cumbersome and are difficult to use. Also, many must be used with direct interaction with a physician or other healthcare provider and, thus, deny the patient mobility when in traction. Because these problems often affect the everyday lives of those who suffer from sever neck pain, a portable traction device that can be used while the person goes about his daily tasks can prove to be a far better solution.

A method commonly used by chiropractic professionals for treatment of patients with neck pain involves restraining the patient in a retractor device and stretching their cervical spine along with a physical therapy regimen. It is common for this technique to relieve neck pain for a short period of time and is not effective as a long term palliative treatment of the pain.

While technology has resulted in our ability to produce instruments, devices and pharmaceuticals for treatment of spinal pain, there is still a clear need for an apparatus that would relieve the body weight on spine while allowing the patient to have normal motion in the horizontal, sagital and coronal body planes in a standing or sitting position. It is further desired for this apparatus to be low profile. Additionally, any intervention that would hasten the conservative recovery process, such as by facilitating independent patient participation in his/her own care, should also result in significant healthcare cost savings.

There are a number of known methods and devices for providing traction of the spine each focusing on the neck or the back. Devices and methods focused on providing traction to the cervical spine are separated from the devices for the thoracic and lumbar spine.

### A. Current Neck Traction Devices

For the cervical column or the neck, US patent publication US 2007/0106194 to Pickering discusses a traction device having an adjustable tensioning mechanism; the traction device is attachable to a head support and a cervical collar. The tensioning mechanism includes a traction base, a slider connected to the traction base and an adjustment mechanism to force the traction base and slider in opposite directions. One of the traction bases or sliders is at a fixed longitudinal position with respect to the head support and the other traction base or slider is at a fixed longitudinal position with respect to the cervical collar. The Pickering device does not allow a patient to have free motion of the neck with respect to the rest of the body and although it allows the patient to be mobile and walk around freely, it does not allow mobility of the head with respect to spine. Moreover, the Pickering device does not provide full mobility of a patient's head and is not suitable for the everyday activities of a neck pain patient.

Neck traction systems also exist where a patient wears a device that circles under their chin and attaches the device to an elevated point, such as the top of a door to provide traction. Such devices lack mobility and comfort, and rely on patient compliance to be effective.

Mechanical or pneumatic devices also exist for cervical spine fraction such as the Saunders Hometrac™ and the Pronex®. These devices are neither mobile nor allow patient mobility. To use a pneumatic device the patient is typically in a prone position. Air-inflated bellows are used to create varying degrees of traction. The patient must be stationary during an extended period of time for the fraction to be effective.

Previous mechanical efforts to support the cervical spine or apply traction thereto have generally fallen into certain distinct design trends. Full, solid cervical collars incorporating single or multiple, stacked pneumatic/air bladders are shown in the following United States patents: Pillai, U.S. Pat. No. 6,050,965; Rogachevsky, U.S. Pat. No. 5,752,927; Bragg, U.S. Pat. No. 5,403,266; Ommaya, U.S. Pat. No. 3,765,412; and Zumaglini, U.S. Pat. No. 3,343,532. Full, solid cervical collars incorporating multiple, stacked, fluid-filled bladders are shown in Park, U.S. Pat. No. 5,823,982. A full, but open, cervical collar, attached to a heavy chest/back apron/harness by adjustable spring-loaded rods is shown in Grober, U.S. Pat. No. 6,045,522, whereas McFarland, U.S. Pat. No. 3,776,224 shows a similar spring-tensioned device. Hanicke, U.S. Pat. No. 2,102,069 shows spaced pads carried by independent side braces that are angularly adjustable and are also longitudinal adjustable via sliding members and clamping screws. Threaded rod adjusters are shown in Hale, U.S. Pat. No. 2,736,314, in Hall, U.S. Pat. No. 2,820,455, and in Bartels, U.S. Pat. No. 3,177,869. Young, U.S. Pat. No. 5,046,490 places a peg in a hole on a sliding bar to fix the length and employs a hinge mechanism to control abduction and abduction in one embodiment and, in another, uses nuts on a threaded rod to cause a collar to slide up and down. A rack and pinion system is shown in Bustamante, U.S. Pat. No. 2,791,999. A halo to skull fixation device is attached to shoulder harness by calibrated threaded rods in Bode, U.S. Pat. No. 5,195,947 and in Gullichsen, U.S. Pat. No. 5,697,894. A full collar with a tracheal core aperture designed for static support and emergency immobilization is shown in Calabrese, U.S. Pat. No. 4,886,052, and a full collar with a ratchet and pawl mechanism for improved fit, emergency immobilization is shown in Martin, U.S. Pat. No. 6,036,664.

U.S. Pat. No. 5,651,754 to Chiu shows a portable device for reforming the spine which utilizes a belt that is tightened about the waist and a motor-driven vertical rod that spaces a brace that engages the arm pits or the chin. In spite of their claims to the contrary, none of the above-referenced devices provides the patient with a truly user-friendly, simple, lightweight, easily installed and easily adjusted device which allows the application of appropriate, effective and efficient cervical traction by the patient in any setting, e.g. while the patient is an upright, mobile position. As a result, the search has continued to construct a device having these attributes.

### B. Current Back Brace Devices

The devices for relief of back pain includes braces, splints, and supports but none that address the above-described limitations. For example, U.S. Patent Publication US 2005/0010150 to Firsov discusses an orthopedic device with adjustable support and forces for musculoskeletal disorders by placement of axial members in a wearable device. The Firsov device does not relieve upper body weight through the spine. U.S. Pat. No. 5,613,941 discloses a joint support apparatus for wrapping around a joint to protect and support the joint. Such an apparatus removes loads and supports the joint while allowing motion, but immobilizes the joint firmly and no actively imparting forces to move the joint and may be applicable to varying individual joints but does not relieve pressure of the upper weight on the lower body. U.S. Pat. No. 6,142,965 discloses a brace constructed from flexible material designed to restrict rotational movement about a joint, which provides active resistance to rotation. Such a device is applicable only to a limb and does not relieve back pain due to axial pressure of the upper body and does not actively correct postural problems. Devices such as those disclosed in U.S. Pat. No. 5,967,998 and U.S. Pat. No. 6,213,968 are orthopedic braces which support the torso and spine (although only the lumbo-sacral and not the thoracic and cervical), and are adjustable; however, the adjustments are only to allow the brace to conform to the torso even with changes in position of the wearer and not to permit the application of manipulating forces. They are only for stabilization and do not transfer compressive weight of the upper body to the hip and therefore do not relieve upper body weight forces on the spine. They do not allow specific localization of restriction of movement either: for example, allowing rotation only to the right at the level of L1 to L5 without restricting movement at other levels. The inflatable wearable traction device disclosed in U.S. Pat. No. 5,950,628 suffers from similar limitations.

U.S. Pat. No. 6,213,922 to Afanasenko et al. is directed to a device for treatment of patients with disturbed posture and motor activity comprising shoulder, pelvic, knee, pedal, elbow, hand, and finger supports, all of them interconnected by fixing elements, which are shaped as elastic tie-members and placed on the surface of the patient's body in antagonistic pairs. Each tie member is connected to two supports and includes a tension adjuster. The Afanasenko et al. device does not relieve the impact of upper body weight from the spine structure. Moreover, the tension adjustment is not made in real-time as a patient moves therefore not allowing full motion of the upper body. A vertical upper body support for retracting the spine capable of adjusting to modify the force and direction of support is not provided. Full rigid support of the upper body is not possible. Creation of support along the length of a spine is not possible. Each support is separately applied making application difficult.

What is needed is a device and method that will enable people with spinal pain to be relieved of head weight or upper body weight through the spine structure and therefore may relieve pain associated with the spine yet maintain normal physical mobility of the body.

### SUMMARY OF THE INVENTION

The devices, methods and systems described are used in producing devices for relieving cervical, or neck pain and for relieving lumbar pain, or lower back pain and for relieving thoracic pain, upper spine pain or neck pain. The devices according to this disclosure allow for the free motion of the body in all three body planes while transferring the downward force applied by weight of the body above the device to anatomy below the device and thereby relieving pressure on the targeted anatomy surrounded by the device or applying enough force against the body to retract the target area of the spine while allowing full and independent motion of the body with respect to the rest of the body.

An aspect of the disclosure is directed to an orthopedic device that transfers downward force away from a target anatomy. A device comprises: a conformable upper member having a first end and a second end and a length sufficient to circumvent a target anatomy; a conformable lower member having a first end and a second end and a length sufficient to circumvent a target anatomy; an upper member locking mechanism adapted and configured to releasably connect the first end of the upper member to the second end; a lower member locking mechanism adapted and configured to releasably connect the first end of the lower member to the second end; one or more rods configured to connect the upper member to the lower member wherein the one or more rods pivotally connect at a first end to the upper member and at a second end to the lower member to a respective connection member. Devices can additionally be configured to provide a support member, or a support member having an adjustable length and/or a release mechanism. Additionally, the devices can be configurable to include a sacrum member and/or a shoulder blade member. The sacrum member and should blade member can further be removeable, if desired. Adjustment mechanisms can also be provided that enable the length of the upper member and lower member to be adjusted. Locking mechanisms can be a buckle having a male end, a female end and a release mechanism, Velcro®, or any other suitable locking mechanism. Padding may also be provided on all or parts of the device. Padding may particularly be provided at locations on the device where the device comes into contact with the anatomy. Typically at least one of the upper member and the lower member further include a conformable compliant plate, such as a plate configured from suitable shape memory material (e.g., nitinol). Additional configurations include one or more rods or one or more leaf springs. Moreover, the one or more rods can be configured such that they are tensioned. The one or more rods can be installed in pairs where the ends of the rods have a pivotal connection to the upper member at a single location and a pivotal connection to the lower member at a single location. In other configurations, one of the two upper and lower connections can be fixed while the remaining connection is pivotally engaged. The devices can be configured to have upper and lower conformable sections that are adapted and configured to conform to the anatomy adjacent the device. Thus, for example, the upper section can be adapted and configured to conform or be conformable to the chin and the back of the skull and a lower section that is adapted and configured to conform or be conformable to the back and clavicle. In other configurations, the upper member can be adapted and configured to conform or be conformable to the chest and underarms with a lower member adapted and configured to confor or be conformable with the hips.

Other aspects of the disclosure relate to methods for treating and/or alleviating spinal pain. Methods comprise: wearing a device comprising a conformable upper member having a first end and a second end and a length sufficient to circumvent a target anatomy, a conformable lower member having a first end and a second end and a length sufficient to circumvent a target anatomy, an upper member locking mechanism adapted and configured to releasably connect the first end of the upper member to the second end, a lower member locking mechanism adapted and configured to releasably connect the first end of the lower member to the second end, one or more rods configured to connect the upper member to the lower member wherein the one or more rods pivotally connect at a first end to the upper member and at a second end to the lower member to a respective connection member such that the upper member of the device is positioned at or above a target anatomy and the lower member of the device is positioned at or below a target anatomy; transmitting a downward force from an anatomy at or above the upper member to an anatomy at or below the lower member, wherein wearing the device does not immobilize the wearer. Additional steps include applying traction to an anatomy at or below the upper member and at or above the upper member, connecting the device to at least one of a helmet and one or more shoulder pads, and/or applying a force that pulls the upper member and the lower member towards each other via the vertical support and applying a force that pushes the upper member and the lower member away from each other via the rods.

Still other aspects of the disclosure relate to a system for relieving pain or applying traction to one or more areas of the spine. Suitable systems comprise: a first device having a conformable upper member having a first end and a second end and a length sufficient to circumvent a target anatomy, a conformable lowermember having a first end and a second end and a length sufficient to circumvent a target anatomy, an upper member locking mechanism adapted and configured to releasably connect the first end of the upper member to the second end, a lower member locking mechanism adapted and configured to releasably connect the first end of the lower member to the second end, one or more rods configured to connect the upper member to the lower member wherein the one or more rods pivotally connect at a first end to the upper member and at a second end to the lower member to a respective connection member configured to fit a first target anatomy, and a second device having a conformable upper member having a first end and a second end and a length sufficient to circumvent a target anatomy, a conformable lowermember having a first end and a second end and a length sufficient to circumvent a target anatomy, an upper member locking mechanism adapted and configured to releasably connect the first end of the upper member to the second end, a lower member locking mechanism adapted and configured to releasably connect the first end of the lower member to the second end, one or more rods configured to connect the upper member to the lower member wherein the one or more rods pivotally connect at a first end to the upper member and at a second end to the lower member to a respective connection member configured to fit a second target anatomy. Additionally, in some configurations of the system the first device and the second device are connected via one or more rods or supports.

### INCORPORATION BY REFERENCE

All publications and patent applications mentioned in this specification are herein incorporated by reference to the same extent as if each individual publication or patent application was specifically and individually indicated to be incorporated by reference.

In addition to other references disclosed in the application, the following references are also of interested in the field of the disclosure:
U.S. Patent Publication 2009/0198163 for Traction Apparatus and Methods by Senyei et al.; U.S. Patent Publication 2007/0106194 for Traction Collar and Method of Use by Pickering, et al.,
U.S. Patent Publication 2005/0010150 for Customizable Orthotic Device with Adjustable Support and Forces by Firsov;
U.S. Patent Publication 2002/0151833 for Cervical Traction Device and Method by Pettibon;
U.S. Pat. No. 7,449,005 for Traction Collar and Method of Use by Pickering, et al.;
U.S. Pat. No. 7,128,724 for Cervical Spine Brace and Traction Device by Marsh;
U.S. Pat. No. 6,500,136 for Cervical Remodeling Collar by Meyer;
U.S. Pat. No. 6,368,295 for Non-Invasive Halo-Type Cervical Brace by Lerman;
U.S. Pat. No. 6,213,968 for Custom Fitted Orthotic Device by Heinz, et al.;
U.S. Pat. No. 6,213,922 for Device for Treatment of Patients with Disturbed Posture and Motor Activity by Afanasenko, et al.;
U.S. Pat. No. 6,142,965 for Variably Adjustable Bi-Directional Derotation Bracing System by Mathewson;
U.S. Pat. No. 6,050,965 for Cervical Collar for Lifting the Skull of a Wearer by Pillai;
U.S. Pat. No. 6,045,522 for Cervical Spine Traction Apparatus by Grober;
U.S. Pat. No. 6,036,664 for Automatic Adjustable Cervical Collar by Martin, et al.;
U.S. Pat. No. 5,967,998 for Lumbo-Sacral Orthosis by Modglin;
U.S. Pat. No. 5,950,628 for Inflatable Wearable Traction Device by Dunfee, et al.;
U.S. Pat. No. 5,823,982 for Traction Apparatus for Physical Therapy of Herniated Nucleosus Pulposus or Sprain and Strain by Park;
U.S. Pat. No. 5,752,927 for Inflatable Cervical Traction Device by Rogachevsky;
U.S. Pat. No. 5,613,941 for Joint Support Apparatus by Prengler;
U.S. Pat. No. 5,403,266 for Inflatable Cervical Traction Collar by Bragg, et al.;
U.S. Pat. No. 5,195,947 for Calibrated Cervical Traction Device by Bode;
U.S. Pat. No. 5,046,490 Orthopaedic Brace and Motion Control Mechanism Therefore by Young, et al.;
U.S. Pat. No. 4,886,052 for Emergency Medical Cervical Collar by Calabrese;
U.S. Pat. No. 3,776,224 for Dynamic Cervical Support by McFarland;
U.S. Pat. No. 3,765,412 for Inflatable Cervical Collar for Prevention of Head and Neck Injury by Ommaya;
U.S. Pat. No. 3,343,532 for Orthopaedic Apparatus for Immobilizing and Stretching the Cervical Column by Zumaglini;
U.S. Pat. No. 3,177,869 for Cervical Neck Brace by Bartels;
U.S. Pat. No. 2,791,999 for Neck Exercising Device by Bustamante;
U.S. Pat. No. 2,736,314 for Cervical Brace by Hale; and
U.S. Pat. No. 2,102,069 for Cervical Splint by Hanicke.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1a is a lateral elevation view of a normal human spinal column;

FIG. 1b is a perspective view of the anatomical planes of the human body;

FIG. 2a is a perspective view of a devices suitable for use in the lumbar-thoracic spine;

FIG. 2b is a flat interior view of the lumbar-thoracic spine device of FIG. 2a;

FIG. 2c is a flat exterior view of the lumbar-thoracic spine device of FIG. 2a;

FIG. 2d is a front view of a human wearing the lumbar-thoracic spine device of FIG. 2a;

FIG. 2e is a rear view of a human wearing the lumbar-thoracic spine device of FIG. 2a;

FIG. 3a is a perspective view of a devices suitable for use in the cervical spine;

FIG. 3b is a flat interior view of the cervical spine device of FIG. 3a;

FIG. 3c is a flat exterior view of the cervical spine device of FIG. 3a;

FIG. 3d is a front view of a human wearing the cervical spine device of FIG. 3a;

FIG. 3e is a rear view of a human wearing the cervical spine device of FIG. 3a;

FIG. 4a is a perspective view of a devices suitable for use in the lumbar spine;

FIG. 4b is a flat interior view of the lumbar spine device of FIG. 4a;

FIG. 4c is a flat exterior view of the lumbar spine device of FIG. 4a;

FIG. 4d is a front view of a human wearing the lumbar spine device of FIG. 4a;

FIG. 4e is a rear view of a human wearing the lumbar spine device of FIG. 4a;

FIG. 5a is a perspective view of a devices suitable for use in the lumbar spine;

FIG. 5b is a flat interior view of the lumbar spine device of FIG. 5a;

FIG. 5c is a flat exterior view of the lumbar spine device of FIG. 5a;

FIG. 5d is a front view of a human wearing the lumbar spine device of FIG. 5a; and

FIG. 5e is a rear view of a human wearing the lumbar spine device of FIG. 5a.

### DETAILED DESCRIPTION OF THE INVENTION

This disclosure relates to devices for stretching the human spine and relieving the weight and the compressive force that is applied, e.g. in the caudad direction, to the spine by transferring the downward force applied by weight above one section of the spine to anatomy below a target section of the spine and thereby by passing all or part of the resultant force of weight of a section of body from a target area of the spine to another area of the anatomy. Removal or reduction of the compressive force due to the weight of the transferred section may relieve spine pain because the full compressive forces on the spine are no longer present, similar to when a person with severe neck pain lays down on their back in order to hold their spine in a flat relaxed position. The devices of this disclosure may also be used for treatment of spine deformities in outer space where gravity is absent and bones soften. Additionally, the devices of this disclosure can be used to prevent or minimize cervical fractures that result from force applied during a severe blow to the head. For example, sports involving violent physical contact carry a risk of cervical fracture, including American football, ice hockey, rugby and wrestling. Although other non-contact sports also have arisk of cervical fracture, including, for example, skiing, diving, surfing, powerlifting, equestrianism, mountain biking and motor racing.

In another application of the device, patients having undergone spinal fusion may benefit from wearing the device. Approximately 19% of spinal fusion patients experience adjacent segment degeneration (or adjacent spine degeneration). This condition typically develops over a period of five years from the surgery.

### A. Anatomical Review

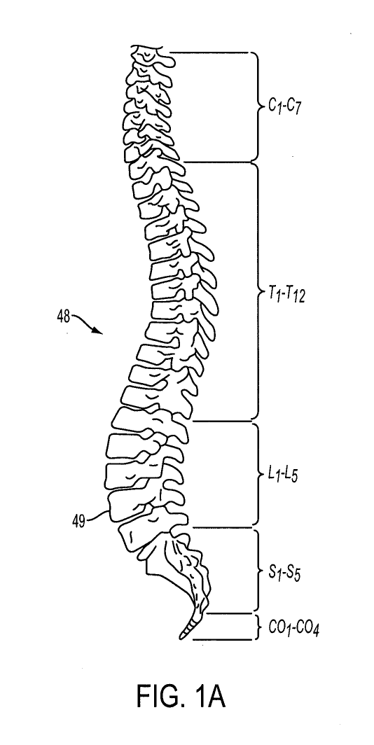

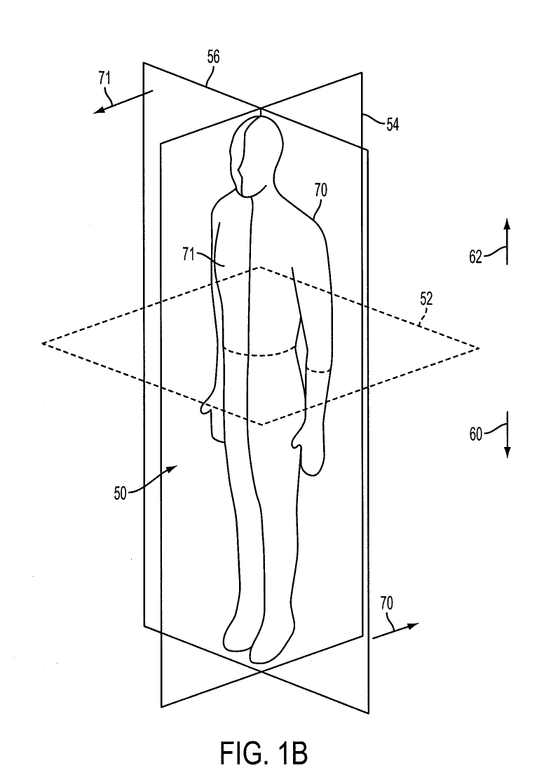

Devices and their usefulness can be illustrated in the context of spinal pathologies. In order to appreciate the usefulness of the devices enabled by this disclosure it is helpful to understand an anatomical environment where the devices can be used. Thus, for example, the devices are adapted and configured to interact with the human spinal column 48, as shown in FIG. 1a, which is compromised of a series of thirty-three stacked vertebrae 49 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4. Although human lumbar vertebrae vary somewhat according to location, the vertebrae share many common features.

The spinal cord is a long, thin, tubular bundle of nerves that is an extension of the central nervous system from the brain. The spinal cord is positioned in the vertebral foramen and protected by the bony vertebral column. The main function of the spinal cord is transmission of neural inputs between the periphery and the brain. Three meninges cover the spinal cord: the outer dura mater, the arachnoid mater and the innermost pia mater. Cerebrospinal fluid is found in the subarachnoid space and the spinal cord is stabilized within the dura mater by the connecting denticulate ligaments which extend from the enveloping pia mater between the dorsal and ventral roots. The lamina provides protection for the dural sac and a foundation for the spinous processes. And epidural space is provided between the spinal cord and the vertebral arch defining the vertebral foramen. A portion of the vertebral foramen is also occupied by the ligamentum flavum. The ligamentum flavum connects the lamina of adjacent vertebra. However, the ligamentum flavum can become thickened, thereby reducing the cross-sectional volume in the vertebral foramen available to house the spinal cord. As a result pressure is applied to the spinal cord resulting in back pain, numbness of the legs, etc.

In order to understand the configurability, adaptability, and operational aspects of the disclosure and methods, it is helpful to understand the anatomical references of the body 50 with respect to which the position and operation of the devices, and components thereof, are described as shown in FIG. 1b. There are three anatomical planes generally used in anatomy to describe the human body and structure within the human body: the axial plane 52, the sagital plane 54 and the coronal plane 56. Additionally, devices and the operation of devices and tools are better understood with respect to the caudad 60 direction and/or the cephalad direction 62. Positioning or operation of the device can be dorsally 70 (or posteriorly) toward the back or rear of the body. Alternatively, positioning or operation of the devices can be ventrally 72 (or anteriorly) such that the placement or operation of the tool or device is toward the front of the body.

The functional unit of the spine is composed of two adjacent vertebral bodies, two facet joints, an intervertebral disk and associated ligaments. The primary function of the vertebral disc in the spine is to distribute the load of the body above it through the length of the spine.

With time and trauma, the discs progressively degenerate and become internally disrupted. If this internal disruption allows protrusion of the disc it is called disc herniations and when the disc material reaches the epidural space it may cause an inflammatory reaction in a spinal nerve leading to acute neck, back or extremity pain. This damage of the discs, facets and ligaments with trauma and/or aging may lead to narrowing of the neural foramina through which all spinal nerves pass, a condition known as spinal stenosis leading to reticular pain and neurogenic claudication in the neck, back, or upper or lower extremities. Patients with prior spine surgery may develop recurrent spine pain. They represent a failed back surgery syndrome.

Treatment of cervical spine pain consists of several methodologies. For cervical spine as an example, there must be a diagnosis as to whether the pain is in the neck or does it radiate into the upper extremities. Most acute, non-reticular neck pain is secondary to traumatic sprain/strain of muscles, ligaments and facet joints. These patients usually recover within three months treated with physical therapy, mild analgesics and muscle relaxants. Neck and upper extremity pain lasting longer than three months is considered to have become chronic and invasive measures may be needed.

Acute spine pain associated with pain radiating into the arms or legs, is a condition known as reticular neuropathic pain and warrants more aggressive therapy. This may include strong neuropathic medications such as Lyrica® (pregabalin) available from Pfizer and even opioids such as buprenorphine (Buprenex®), butorphanol (Stadol®), codeine (Tylenol® with codeine), fentanyl (Duragesic®), hydrocodone (Vicodin®), hydromorphone (Dilaudid®), methadone (Dolophine), oxycodone (OxyContin®), and propozyphene (Darvon). As is well known in the art, medications are not without potential side effects including, but not limited to, addiction.

One type of aggressive but non invasive, non-drug therapy that has been recommended is spine bracing and stretching, commonly known as distraction of the spine. These modalities can be accomplished either in a practitioner's office, for example, physical therapist and/or chiropractor. Currently, there are no commercial devices available that provide adequate modalities effectively, while allowing the patient good spine mobility and continued participation in work and leisure activities.

The idea behind stretching or distracting the spine in patients with spine pain radiating into the extremities is that spinal nerves have been compromised by foramina narrowing causing spinal stenosis or ruptured discs. This leads to distraction of the spine relieving the compression and allowing healing of the nerve. Intermittent distraction, lasting minutes or hours, often provides temporary relief of the radicular pain but nothing permanent.

A study by Janus et al, in The Spine, April 2004, Volume 29, Issue 7, pp. 763-766 revealed that minimal posterior distraction of 4 to 6 mm at C5-C6 and C6-C7 in six human cadaver cervical spine specimens enlarged the neuroforamen by 10 to 18 mm. at the same time the distance between vertebral bodies would also be distracted but this was not looked at in the study.

A constant traction gently stretches muscles and ligaments, which allows increased or restored flow and nourishment to the compressed spinal nerves. A device that provides continuous spine distraction in a fully functional patient with radicular pain is not available to the medical community.

Considering the results of the study in human cadavers allows a speculation that constant use of a stretching spine brace would allow some degree of foramen and disc distraction to assist in reduction of radicular pain and perhaps allow healing with more permanent benefit than the present short-term spine distraction.

### B. Devices

The devices of this disclosure may be produced to fit between the human waist and chest in order to treat lumbar-thoracic spine pain or lumbar spine pain or thoracic spine. The devices of this disclosure may also be configured to fit around the human neck in order to treat cervical spine pain. The devices of the disclosure may also be configured to fit between a helmet and shoulders (or shoulder pads). Additionally, two devices can be used in combination, for example, a cervical device can be used in combination with a lumbar-thoracic device. Where two devices are used, the devices may or may not be connected to one another.

(1) Lumbar-Thoracic Devices Adapted to Mitigated Weight and Force While Maintaining Mobility

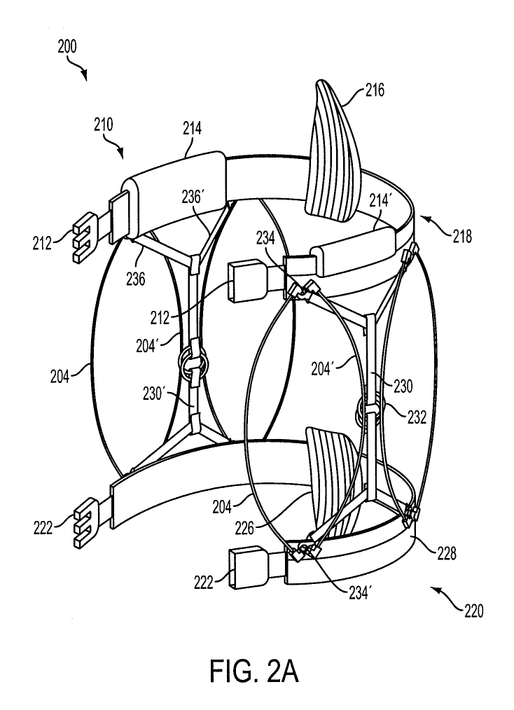

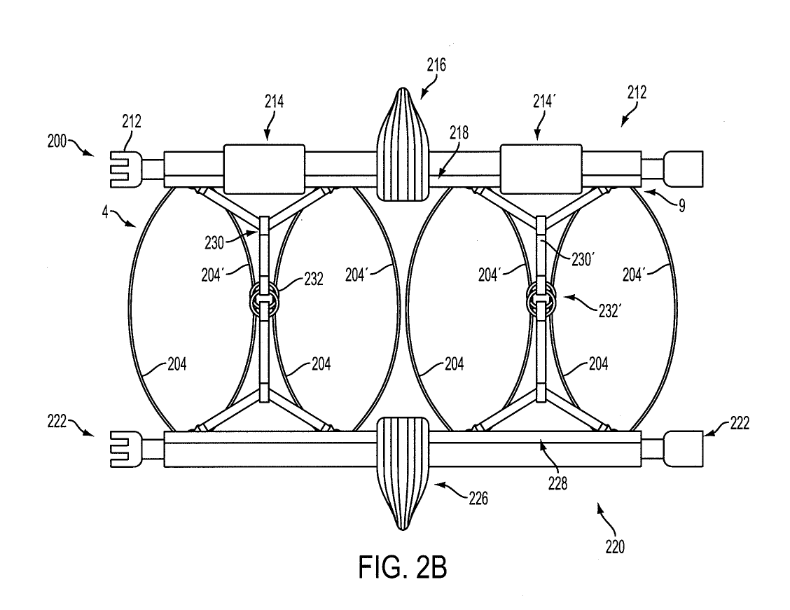

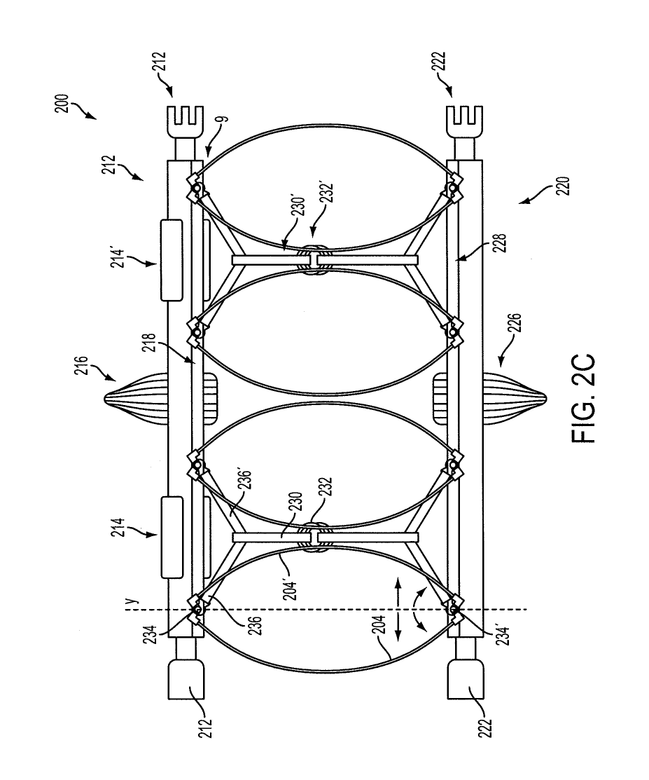

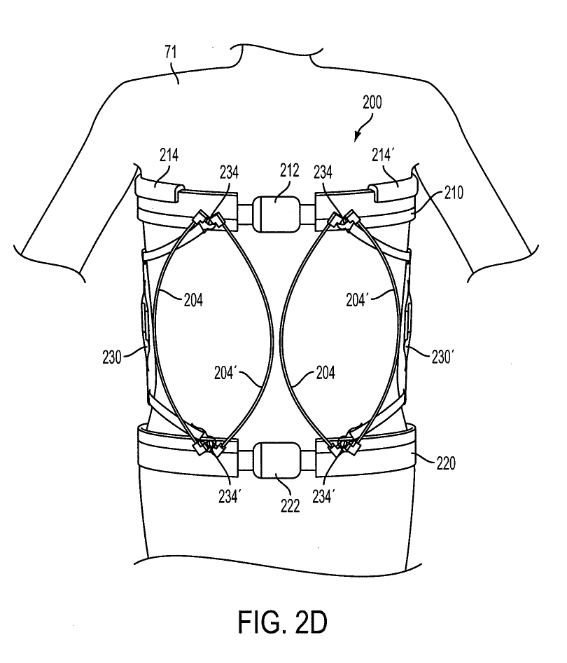

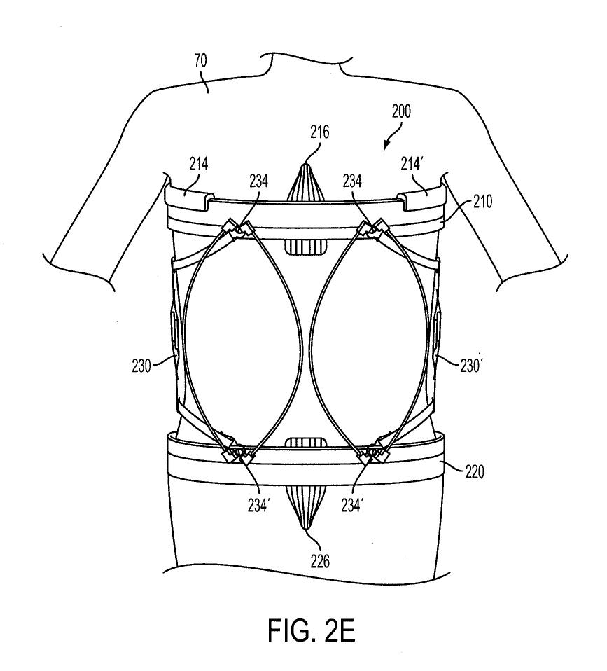

FIG. 2a is a perspective view of a lumbar-thoracic device 200 of this disclosure having an upper belt 210 and a lower belt 220. The belts can be configured from any suitable material including, for example, foam, fabric and metal. Additionally, the belts can be treated with suitable waterproofing compounds (e.g. where foam and/or fabric is used) or anti-microbial material. The upper belt 210 and lower belt 220 are connectable via opposing rods 204, 204′ which are used as leaf springs to provide an expansive force pushing the upper belt 210 away from the lower belt 220. The rods can be made from any suitable material including, for example, shape memory material (such as nitinol), and fiberglass. Opposing rods 204, 204′ are arranged in opposing pairs in this particular design, but it is appreciated that they may be individual or in combination of more than two rods. A first end of each of the opposing rods 204, 204′ connects to the upper belt 210 at a first connection point 205, and a second end of each of the opposing rods 204, 204′ connects to the lower belt 220 at a second connection point 205′. The opposing rods can rotate independently and rotate where fixed to upper and lower belts. This configuration allows rotation in the axial plane. The first and second connection points 205, 205′ can be positioned such that they are in vertical alignment as illustrated. The first and second connection points 205, 205′ can be configured such that they are formed as part of the belt or the plate. In forming the connection points as part of the belt or the plate, the connection points and the belt or plate or both can function as a unified manner.

Additionally, the opposing rods 204, 204′ can be permanently affixed to the connection points 205, 205′ or releasably affixed. Typically, the opposing rods 204, 204′ are engaged at the connection points 205, 205′ such that they are pivotally engaged and allow for asymmetrical pivoting and caudal force transmission. That is, as there is movement (e.g., a person wearing the belt bends forward), one of the opposing rods might curve further away from a vertical axis (which is enabled by pivoting at the connection point) while the second rod becomes straighter. As illustrated here, when the upper belt 210 and the lower belt 220 are positioned parallel one another, the connection rods are positioned along a vertical axis and can be perpendicular to each of the upper belt 210 and lower belt 220 or can form an arc shape away from a vertical axis (shown as y in FIG. 2b.

The upper belt 210 is fitted under the arms of a patient and is fastenable with a suitable fastening mechanism 212. The fastening mechanism can be a buckle type fastener, such as those disclosed in U.S. Pat. Nos. 4,919,484 to Brougher et al. for Anti-Cinch Seat Belt System and 4,832,410 to Brougher for Seat Belt Buckle Assembly. The buckle assembly includes, for example, a buckle and a mounting bracket, a pair of sliding members coupled with one another, and a webbed belt looped through the members and connecting the buckle with the bracket, the sliding members having a first position in which the buckle is in a relatively retracted position and a second position in which the buckle is in a relatively extended position, the assembly including a pair of springs urging the members to the first, retracted position, the application of an external pulling force on the buckle moving the connecting members and buckle against the spring force and into the second, relatively extended position, the connecting members and buckle returning to the first, retracted position upon release of the external force.

Additional padding 214 can be provided to fit under the patient arms and protect the arm pits. The lower belt 220 is similarly fastened about a lower portion of the anatomy, such as the hips, with a fastening member 222. Fastening mechanisms 212, 222 may be “quick release” buckle type mechanism, or “Velcro®” type mechanism requiring, a combination, or another mechanism suitable for securing the device. See, for example U.S. Pat. No. 2,717,437 to deMestral for “Velvet Type Fabric and Method of Producing Same” and U.S. Pat. No. 3,586,060 to Erb for “Method and Apparatus for Making Flexible Strips of Material Having a Pile of Hook-Shaped Elements.” The upper belt 210 and the lower belt 220 are both adapted and configured to have an adjustable length, thereby enabling the device to accommodate a wide variety of patients.

The pivoting connection mechanisms 205, 205′ can be configured such that they are fixed to an upper compliant elongated plate 218 which forms part of upper belt 210. Similarly, a lower compliant elongated plate 228 equipped with pivoting mechanisms 234′ is fixed to the lower belt 220 and is adapted and configured to allow an individual pivot motion of each of the opposing rods 204, 204′. Lower compliant elongated plate 228 and upper compliant elongated plate 218 are flat elongated plates that are compliant enough to wrap about a patient body at a target anatomical location, yet rigid enough to transfer forces transferred from the opposing rods 204, 204′ evenly along the circumference of the upper belt 210 and the lower belt 220. Lower compliant elongated plate 228 and upper compliant elongated plate 218 may be thin aluminum plates, thin plastic plates, or any other material suitable for the device of this disclosure.

As discussed the fastening mechanisms 212, 222 can be in the form of a buckle mechanism with two elongated connectors on a first end 212′, 222′ which are configured to fit within a aperture formed within the receiving housing 212″, 222″ at a second end. Once the two elongated connectors are positioned within the housing, the connectors are releasably locked into place to secure the two ends of the belt 210, 220 together. A release is provided which allows the user to unfasten the mechanism such that the elongated connectors can then be removed from the housing.

A shoulder blade support 216 and a sacrum support 226 can also be provided, as illustrated here, to facilitate positioning the lumbar-thoracic device 200 over the length of the spine and optimizing the location of the device relative to the sacrum and the shoulder blades and to prevent movement of the device in a caudad or cephalad direction (e.g., riding up or down). The shoulder blade support 216 and a sacrum support 226 may be flexible and contoured to fit around the patient shoulder blades and sacrum.

FIG. 2b is the lumbar-thoracic device 200 of FIG. 2a shown in a flat perspective from an interior view. One or more vertical supports 230, 230′ are provided which are adjustably connectable to the upper belt 210 and lower belts 220. A lock/release mechanism 232, 232′ is provided for respective straps which allows the vertical distance between the upper belt 210 and the lower belt 220 to be adjusted. The vertical supports 230, 230′ can be configured to branch 236, 236′ into a Y prior to connecting to either one or both belts. The vertical supports 230, 230′ allow a pull on the upper belt 210 and the lower belt 220 in order to keep the device 200 in a compressed fashion to accommodate placement and removal of the device 200. The lock/release mechanisms 232, 232′ allow locking or releasing of the vertical support straps 230, 230′ and may be configured via any suitable configuration including, for example, a mechanism such as a two-ring lock of motorcycle helmet straps, “quick release” buckles, or “Velcro”, or another mechanism suitable for this function.

FIG. 2c is a flat exterior view of the lumbar-thoracic spine device 200 of FIG. 2a. From this view, the pivot members 205, 205′ can be seen with the opposing rods 204, 204′ and vertical supports 230, 230′ in releasable communication therewith.

FIG. 2d is a front view of a human wearing the lumbar-thoracic spine device 200 of FIG. 2a. The device as configured for this example, fits comfortably around the chest and below the armpits at its upper end and on the hips at its lower end. Two sets of opposing ventral rods 204, 204′ are provided in the coronal plane on either side of a bisecting sagital plane. The locking mechanism 212, 222 is fastened centrally for each of the upper belt 210 and the lower belt 220. However, as will be appreciated by those skilled in the art, the locking mechanism could be positioned dorsally or off-center without departing from the scope of the disclosure. The vertical support members 230, 230′ can be seen positioned vertically down each side of the patient.

FIG. 2e is a rear view of a human wearing the lumbar-thoracic spine device 200 of FIG. 2a. Two additional sets of opposing dorsal rods 204, 204′ are provided in the coronal plane on either side of a bisecting sagital plane. The vertical support members 230, 230′ can be seen positioned vertically down each side of the patient. Additionally, the shoulder blade member 216 and the sacrum member 226 can be seen extending from the upper belt 210 in a cephalad direction (for the shoulder blade member) and from the lower belt 220 in a caudad direction (for the sacrum member).

(2) Cervical Devices Adapted to Mitigate Weight and Force While Maintaining Mobility

Relieving cervical or neck pain is achieved by transferring the force resulting from the weight of the head through an external vertical spring structure to the shoulders thereby relieving pressure on the cervical spine. Further, this device may be used for correcting neck posture by retraction of the neck and therefore the cervical spine. The cervical device can be manufactured in a light-weight design that is particularly suitable for longitudinal traction of the neck for long-term maintenance of cervical anatomy while allowing full mobility of the head with respect to the rest of the body. Advantageously, this dynamic traction device does not require the patient to be lying down, stationary, or holding their head in a stationary position with respect to the rest of the body. The traction device can easily be placed on a patient and adjusted to a desirable force determined by the patient and/or the practitioner. The amount of tension distraction achieved may also be greater than conventional devices. The device holds onto a patient's anatomy underneath the mastoid processes and the mandible and applies a force symmetrically distributed around the cervical spine. This allows the traction to take hold so it does not slide up along the patient's head, providing the ability to impose larger traction than with conventional devices while allowing the patient to have normal speech.

The device of this disclosure is a wearable frame consisting of a padded upper head brace section that may fit below a patients mandible and under the mastoid processes around the head and is fastened about the head. A lower padded hoop section fits over the patients shoulders, a series of springs that are compressed when the device is worn and fitted on the patients neck, and straps on each side for adjusting tension and keeping the entire device in a compressed fashion for ease of placement and removal. The springs are arranged to apply an imparting expansion force distraction in parallel to the patients' cervical spine around the periphery of the neck and applying enough force between the device shoulder and head hoops to reduce or to remove the weight of the head from the cervical spine thereby reducing compression of the spine. Optionally, spring forces may be arranged to provide enough imparting force so that the patients' neck is always under a constant stretch.

The spring mechanism between the two head and shoulder hoops may be coiled or leaf springs that apply expansive forces parallel to the spine and in close proximity to the patients' upper body. Leaf springs may be rods made up of fiberglass, graphite composite, shape memory material, or other material with a flat or round cross section that apply an outward expansive force when bowed. Leaf springs may rotate where they are fixed to the head and shoulder hoops. This allows rotation of the cervical spine in the axial plane.

The frame may be equipped with straps or another mechanism that can hold the device in a compressed position before it is placed on a patient. Upon placement, the straps are released so that the spring mechanism applies imparting pressure between the two hoops transferring head weight to the shoulders when the patient is standing or sitting.

Spring forces may be selected so that the total force applied to the patients cervical spine is greater than the weight of the head. In which case, the patient's neck will always be under a traction force. The stretching retraction of the neck would straighten the patients' deformed cervical spine by removing the weight of the head and transferring it to the shoulders.

The device may be used for correction of neck posture by selecting the springs so the head weight is partially removed from the spine and transferred to the shoulders, with patients' neck being held in an upright position. Additionally, the device may be produced in different sizes to accommodate patients with different physical and anatomical sizes. The device may be produced with springs of higher or lower spring constants in order to accommodate patients in need of greater of lesser traction of the cervical spine. The springs can be made up of any suitable material configurable to transfer force from one section of the device to another. Suitable material includes, but is not limited to nickel-titanium alloy (Nitinol) or any other shape-memory material, thereby simplifying the device manufacturing. Moreover, the springs can be configured from circular or noncircular loops or any other configuration that achieves the desired result.

For the cervical device, a neck hoop or other component may be provided that is adapted and configured to receive or engage a helmet for a sport activity (such as football). A shoulder hoop may be designed to fit on or engage a shoulder pad. Configuring the cervical device to engage a helmet and/or shoulder pads can provide additional protection of the neck in sports where the cervical column is subjected to axial impact forces by offsetting or reducing the axial impact force.

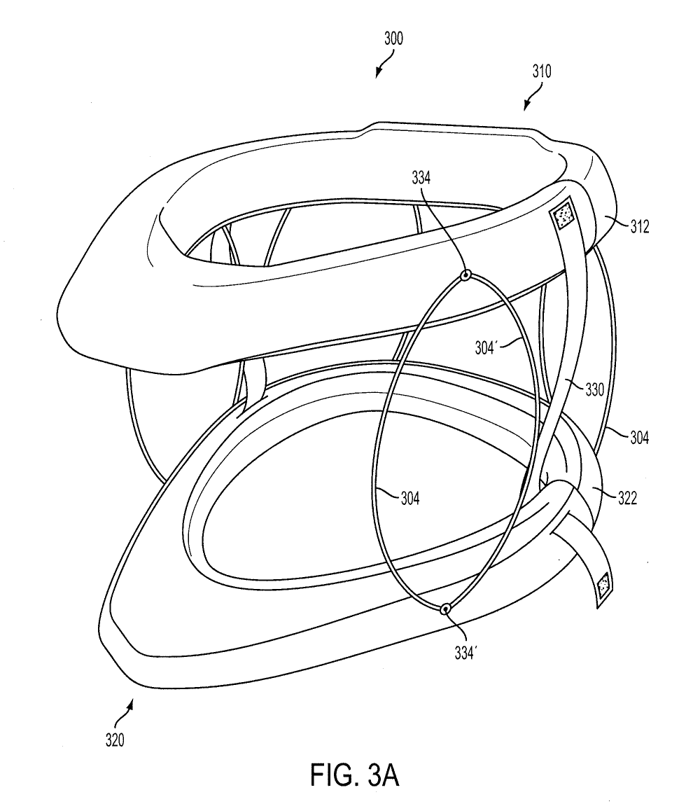

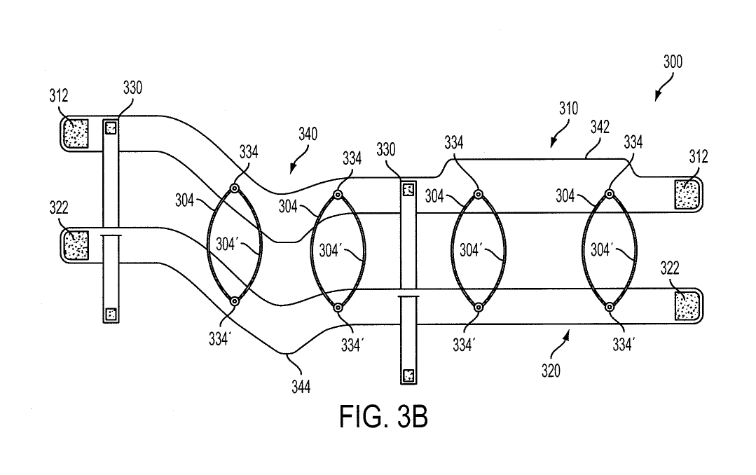

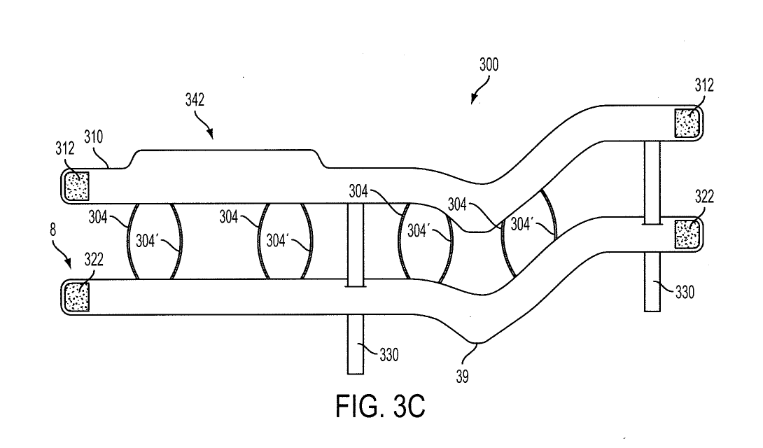

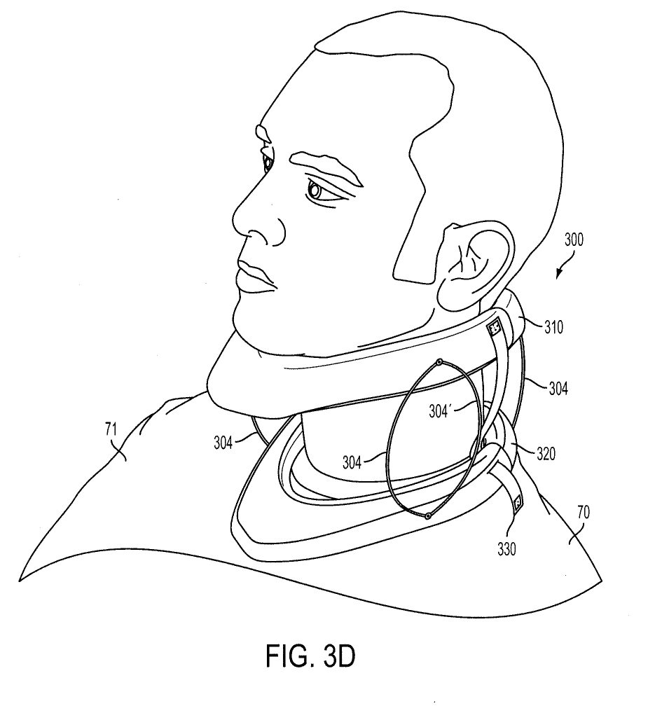

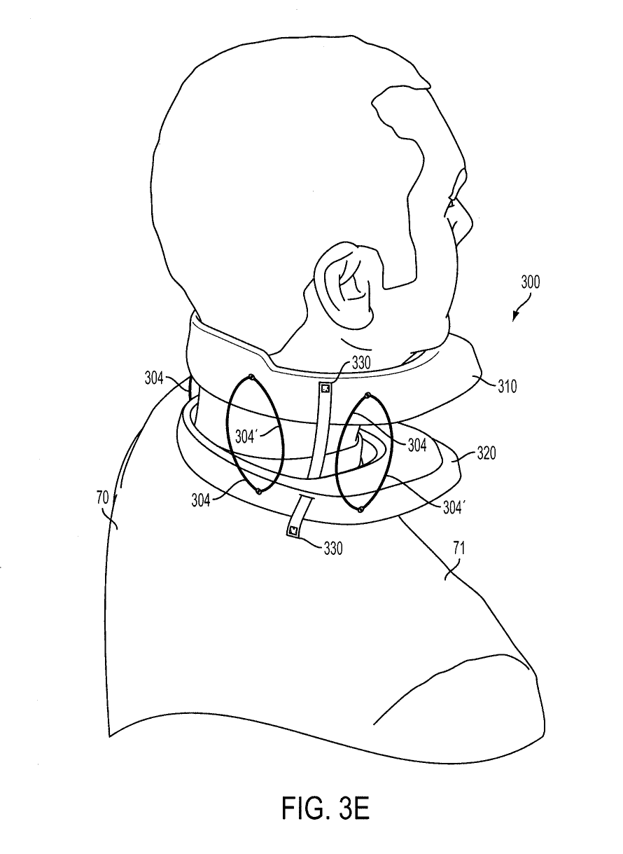

FIG. 3a is a perspective view of a cervical device 300 of this disclosure with opposing rods 304, 304′ used as leaf springs where there is always an expansive force pushing the upper brace 310 away from the lower brace 320. Opposing rods 304, 304′ are arranged in opposing pairs in this particular design, but it is appreciated that they may be individual or in combination of more than two rods, or round loops. The cervical device 300 is fitted under the chin and around the head fitting under the mastoid processes and fastened with a locking mechanism 312 such as Velcro. The lower brace 320 is similarly fastened about the shoulder with a fastening member 322. Upper fastening mechanism 312 and lower fastening mechanism 322 may be “Velcro” type fastening mechanism requiring two mating pieces, or another mechanism. opposing rods 304, 304′ are connect via connection points 305, 305′ on each of the upper brace and the lower brace. The connection points hold the ends of the opposing rods 304, 304′ in place and enable pivoting of the rods about the connection points to accommodate individual bending of each rod symmetrically or asymmetrically relative to the other rod. The leaf spring pairs of the device 300 are duplicated in both braces 2 and 3.

FIG. 3b is a cervical device 300 of this disclosure shown in a flat perspective. The adequately padded upper brace 310 is made of a material that is soft enough to be wrapped about the head yet rigid enough to transfer the leaf spring forces. Cervical device 300 is designed with a section 340 that fits under the chin and a flat section 342 that fits around the back of the head and under the mastoid processes. Velcro mating pieces that form part of the locking mechanisms 312, 322 are mounted on a flexible substrate such as elastic bands commonly used in clothing items. Vertical bands 330, 330′ allow a pull on the upper brace 310 and lower brace 320 in order to keep the device in a compressed fashion in to accommodate placement and removal of the device of the disclosure and allow adjustment of traction forces when worn by the patient. Lower brace 320 is similarly made to upper brace 310 and is designed with a sections 344, 344′ to facilitate fit around the shoulders and around the upper chest.

FIGS. 3d-e show the cervical device 300 of the disclosure as it is worn by a human. The cervical device 300 fits around the head while the lower brace 320 is fixed on top of the shoulders.

(2) Lumbar

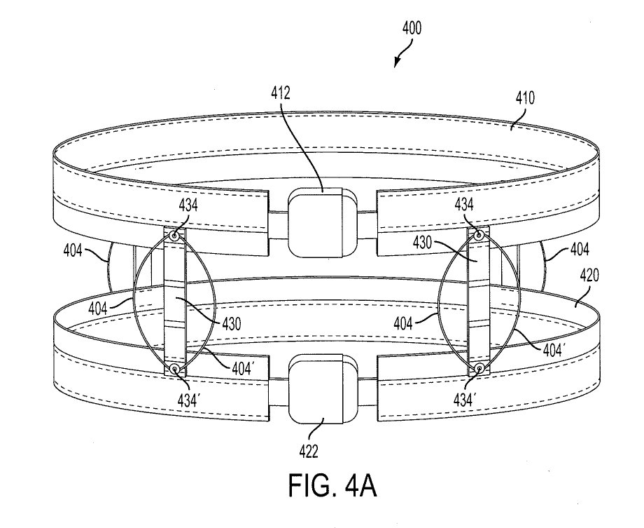

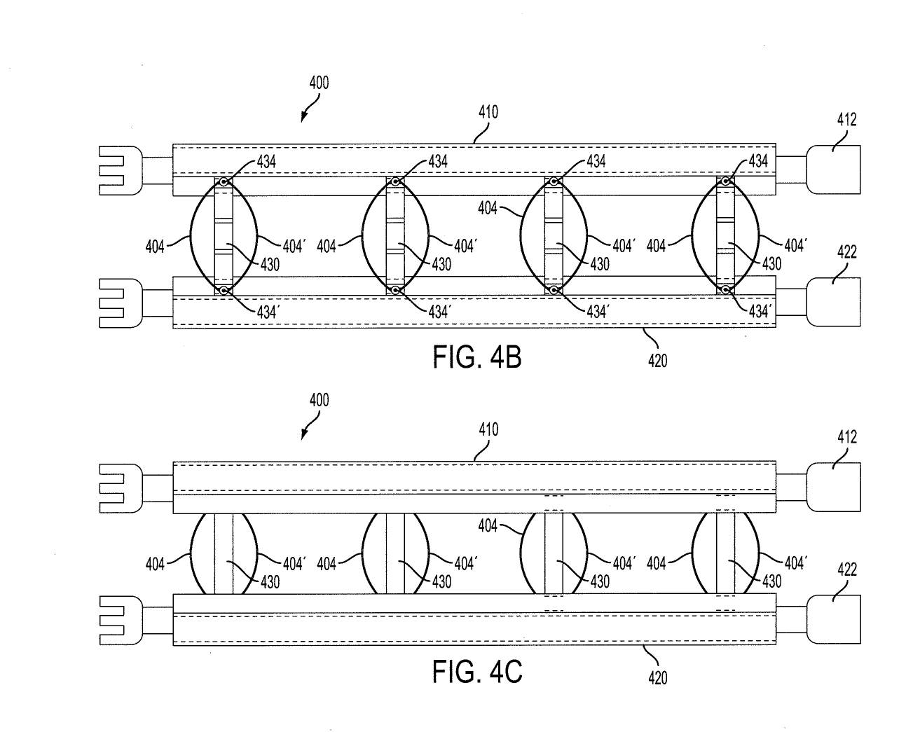

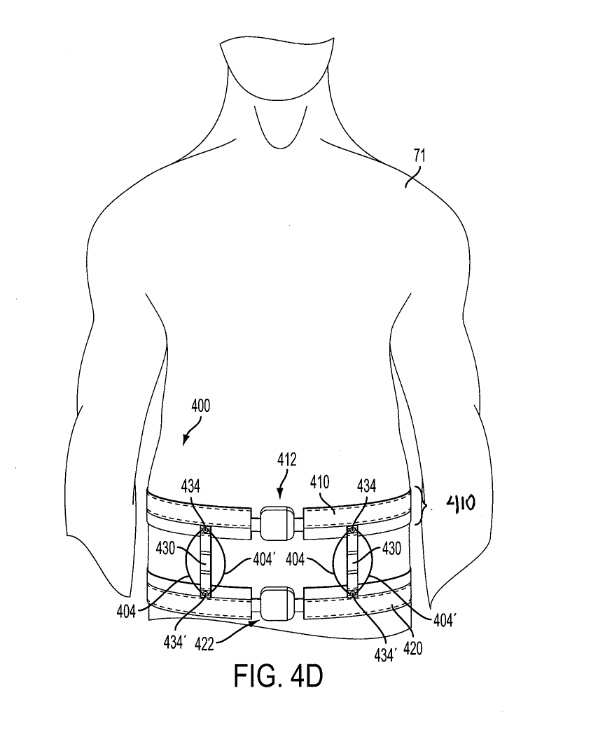

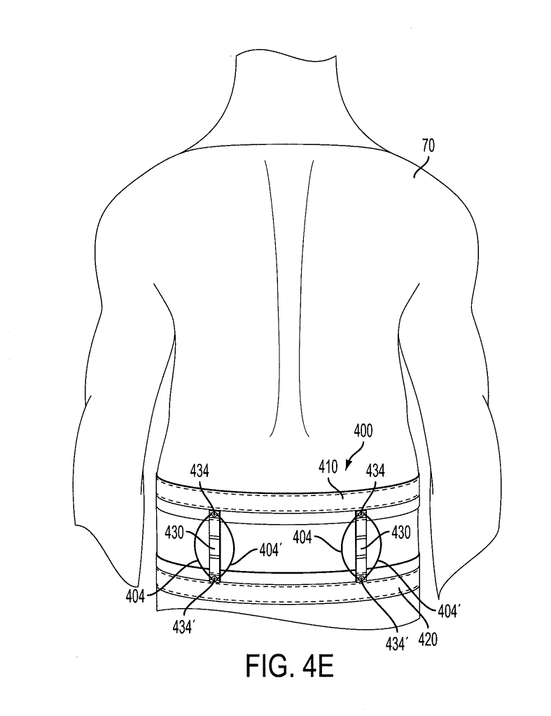

FIG. 4 shows an alternative design of a device 400 of the disclosure. In this design the upper belt 410 rests against the rib cage. Similar to the configuration discussed above for the lumbar thoracic device, the vertical support 430 may be quick release ratchet mechanism or springs achieving the same result as FIG. 2, only focused on affecting the lumbar section of the spine. Rods 404, 404′ provide additional vertical support to remove weight from above the upper belt 410 from being applied to the target anatomy between the upper belt 410 and the lower belt 420.

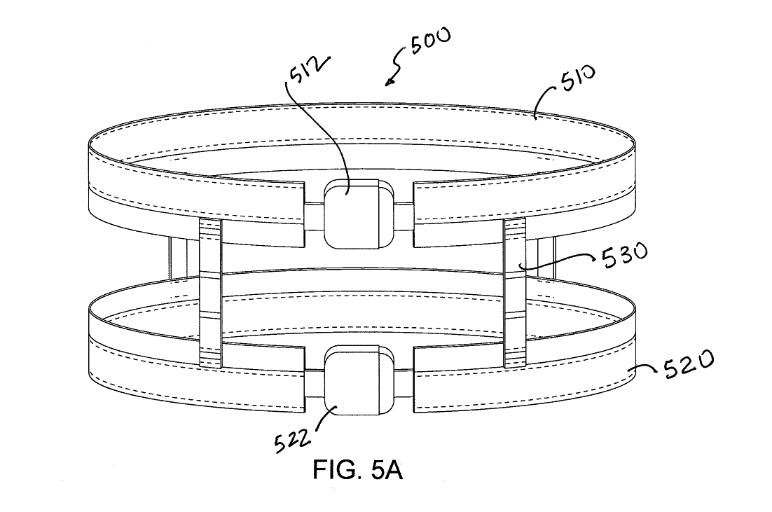

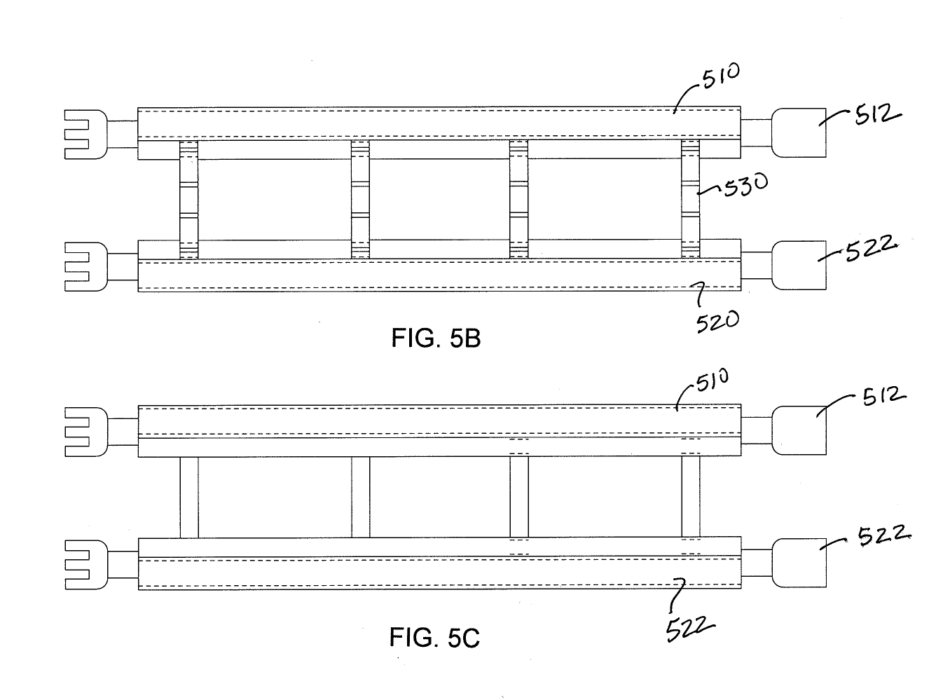

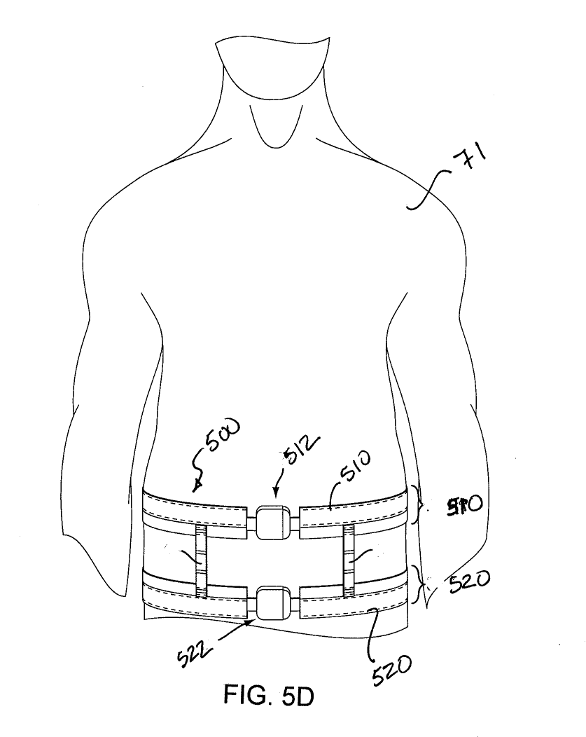

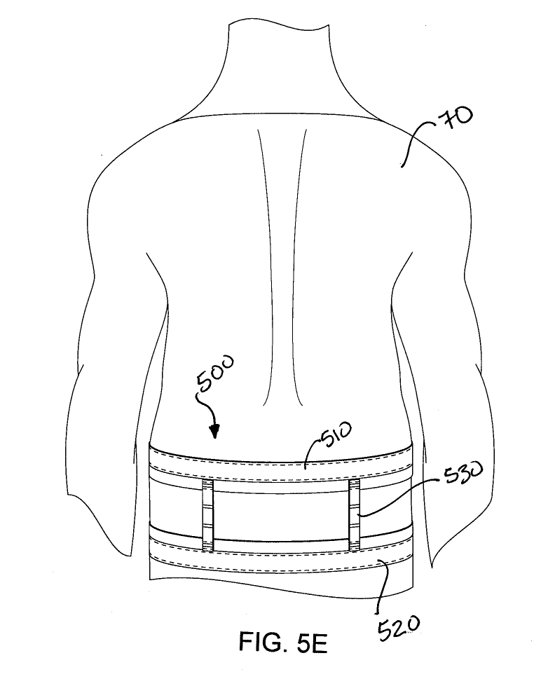

FIG. 5 shows an alternative design of a device 500 of the disclosure. In this design the upper belt 510 rests against the rib cage. Similar to the configuration discussed above for the lumbar thoracic device, the vertical support 530 may be quick release ratchet mechanism or springs achieving the same result as FIG. 2, only focused on affecting the lumbar section of the spine. Rods are not provided. However, the vertical supports 530 can be configured such that the supports achieve the same result as the combination of the vertical supports and the rods. Moreover, in some configurations one or more rods can be incorporated in the vertical supports. As will be appreciated by those skilled in the art, the configurations shown in FIGS. 2 and 3 can also be altered as illustrated herein.

### C. Methods and Examples of Use

A patient wearing a device of this disclosure has their spine held in an upright position. Patients may rotate their spine relative to the body in the horizontal plane with no forces hindering their rotation.

Rotation in and about the sagital, coronal and axial body planes is also possible. As an example, as a patient rotates their head to the right in the sagital plane, the springs on the left side of the neck expand and therefore balance the force requiring compression of the springs to the right of the neck. The same balance is maintained as patients rotate or bend their heads in any direction; therefore, allowing normal motion relative to the rest of the neck in a sitting or standing position. The device of this disclosure assists maintaining neck posture by holding the head in an upright position, removing weight of the head from the cervical spine, transferring the weight of the head to the shoulders, and allowing normal motions of the head relative to the rest of the body.

In another use, a football player wearing a helmet and shoulder pads can be fitted to wear a device illustrated in FIG. 3. So during the blocking maneuver, the player is supposed to keep their head up as they move forward, but it can and does sometimes drop down such that the player is essentially looking at the ground. This position is quite dangerous and can lead to a cervical fracture in the event the player is hit. By providing a brace around the neck, the force applied to the neck can be dispersed, e.g., to the shoulders, thereby reducing the likelihood of a fracture.

### D. Kits

As will be appreciated by those skilled in the art, the devices can be included in a kit. Kit components can include additional straps, additional springs of varying tension to increase or decrease the amount of support for a given deive, thus achieving a customizable support for the patient. Additional springs can be provided to connect one device to another device, where more than one device is worn. Additional components of the kit can include tools necessary to make changes to the devie and/or analgesics or other pharmacological agents that might be part of a treatment protocol.

From the aforementioned description, it is appreciated how the objectives and features of the above-described disclosure are met. The disclosure provides a series of springs between an upper head brace and a lower shoulder brace, the spring force is arranged to stretch between the patients head and shoulders thereby transfering the head weight on the to the shoulders. Springs may also be arranged to stretch the cervical spine. It is appreciated that various modifications of the apparatus are possible without departing from the spirit of the invention.

What is claimed is:

1. A device comprising:
a. a conformable upper member having a first end and a second end and a length sufficient to circumvent a target anatomy;
b. a conformable lower member having a first end and a second end and a length sufficient to circumvent a target anatomy;
c. an upper member locking mechanism adapted and configured to releasably connect the first end of the upper member to the second end;
d. a lower member locking mechanism adapted and configured to releasably connect the first end of the lower member to the second end;
e. one or more rods configured to connect the upper member to the lower member wherein the one or more rods pivotally connect at a first end to the upper member and at a second end to the lower member to a respective connection member.

2. The device of claim 1 further comprising a support member.

3. The device of claim 2 wherein the support member is adjustable in length.

4. The device of claim 2 wherein the support member further comprises a release mechanism.

5. The device of claim 1 further comprising at least one of a sacrum member and a shoulder blade member.

6. The device of claim 1 wherein the upper member and lower member further comprise an adjustment mechanism adapted and configured to adjust the length of the upper member and the lower member.

7. The device of claim 1 wherein the locking mechanism is a buckle having a male end, a female end and a release mechanism.

8. The device of claim 1 further comprising padding.

9. The device of claim 1 wherein at least one of the upper member and the lower member further comprise a conformable compliant plate.

10. The device of claim 9 wherein the conformable compliant plate is formed from a shape memory material.

11. The device of claim 1 wherein the one or more rods are leaf springs.

12. The device of claim 1 wherein the one or more rods are tensioned.

13. The device of claim 1 wherein the one or more rods are at least one pair of rods having a pivotal connection on the upper member at a single location and a pivotal connection on the lower member at a single location.

14. The device of claim 1, wherein the upper member has at least one of an arm conformable section, a breast conformable section, a chin conformable section and an anterior skull conformable section.

15. The device of claim 1 wherein the lower member has at least one of a hip conformable section, a shoulder conformable section, a breast conformable section, and a back conformable section.

16. A method comprising:
a. wearing a device comprising a conformable upper member having a first end and a second end and a length sufficient to circumvent a target anatomy, a conformable lower member having a first end and a second end and a length sufficient to circumvent a target anatomy, an upper member locking mechanism adapted and configured to releasably connect the first end of the upper member to the second end, a lower member locking mechanism adapted and configured to releasably connect the first end of the lower member to the second end, one or more rods configured to connect the upper member to the lower member wherein the one or more rods pivotally connect at a first end to the upper member and at a second end to the lower member to a respective connection member such that the upper member of the device is positioned at or above a target anatomy and the lower member of the device is positioned at or below a target anatomy;
b. transmitting a downward force from an anatomy at or above the upper member to an anatomy at or below the lower member
wherein wearing the device does not immobilize the wearer.

17. The method of claim 16 further comprising the step of applying traction to an anatomy at or below the upper member and at or above the upper member.

18. The method of claim 16 further comprising connecting the device to at least one of a helmet and one or more shoulder pads.

19. The method of claim 16 further comprising the steps of applying a force that pulls the upper member and the lower member towards each other via the vertical support and applying a force that pushes the upper member and the lower member away from each other via the rods.

20. A system for relieving pain or applying traction to one or more areas of the spine comprising:
a. a first device having a conformable upper member having a first end and a second end and a length sufficient to circumvent a target anatomy, a conformable lowermember having a first end and a second end and a length sufficient to circumvent a target anatomy, an upper member locking mechanism adapted and configured to releasably connect the first end of the upper member to the second end, a lower member locking mechanism adapted and configured to releasably connect the first end of the lower member to the second end, one or more rods configured to connect the upper member to the lower member wherein the one or more rods pivotally connect at a first end to the upper member and at a second end to the lower member to a respective connection member configured to fit a first target anatomy, and
b. a second device having a conformable upper member having a first end and a second end and a length sufficient to circumvent a target anatomy, a conformable lowermember having a first end and a second end and a length sufficient to circumvent a target anatomy, an upper member locking mechanism adapted and configured to releasably connect the first end of the upper member to the second end, a lower member locking mechanism adapted and configured to releasably connect the first end of the lower member to the second end, one or more rods configured to connect the upper member to the lower member wherein the one or more rods pivotally connect at a first end to the upper member and at a second end to the lower member to a respective connection member configured to fit a second target anatomy.

21. The system of claim 20 wherein the first device and the second device are connected via one or more rods or supports.
